(19) United States
(12) Patent Application Publication
(10) Pub. No.: US 2009/0131894 A1
(43) Pub. Date: May 21, 2009

(54) Disposable diaper with a unitized double cuff

(76) Inventors: Edward P. Erdman, Westchester, PA (US); Mariela Muniz, West Chester, PA (US)

Correspondence Address: GOSZ AND PARTNERS LLP, ONE STATE STREET, BOSTON, MA, 02109, US

(21) Appl. No.: 11/527,218

(22) Filed: Sep. 26, 2006

(51) Int. Cl. A61F 13/15

(52) U.S. Cl. 604/365; 604/385.28

(57) ABSTRACT

The present invention improves the mechanism of blocking leakage of exudates from a diaper by providing a unitized double cuff along side edges of the diaper. The unitized double cuff comprises a liquid impermeable ribbon bonded to the diaper along its width with two bonds forming an inverted “V” structure and a fringe width of the ribbon extending beyond the inverted “V” structure. This unitized double cuff sticks tightly to a wearer's body and blocks exudates from leaking out of the diaper irrespective of the quantity of exudates or body posture of the wearer.

### BACKGROUND OF THE INVENTION

The present invention relates generally to disposable absorbent articles. More specifically, the invention relates to disposable diapers with optimal means for preventing leakage of body exudates.

In a disposable diaper, an absorbent core placed between a top sheet and a back sheet is used for absorbing body exudates. Typically, fluff pulp has been used as main material for the absorbent core. In order to improve the absorbability of the absorbent core, various measures have been employed such as mixing highly absorptive polymer particles in the pulp. However, the absorbing rates of the absorbent core are not adequately high to absorb a large quantity of body exudates at once, i.e. as soon as the body exudates are excreted onto the upper surfaces of the top sheet and the absorbent core. As a result, the exudates, at least partially, flow onto the upper surface of the topsheet laterally, and often leak out along the side edges of the diaper. Such leakage readily occurs when the quantity of exudates is large.

In order to prevent leakage of exudates from the disposable diaper, leakage protecting baffles or cuffs are provided along the side edges of the diaper. These cuffs conform the side regions of the diaper to the wearer's body thereby preventing exudates from passing out of the diaper. However, diapers with such cuffs have numerous disadvantages. Body exudates may leak out from gaps formed between the cuffs and a wearer's body, for example, when the wearer assumes a certain posture. Moreover, the exudates get accumulated at the cuffs, thereby frequently damaging the cuffs.

U.S. Pat. No. 4,579,556 discloses a disposable diaper in which the opposite side edges of the absorbent core are covered with part of an impervious back sheet, and thereby a certain degree of a leakage protective effect is achieved. However, there is a possibility of excreted material leaking out when the side edges are subjected to body pressure.

U.S. Pat. No. 4,892,528 discloses a disposable diaper in which two sheets resistant to the passage of water are disposed adjacent to and partially outwardly from two side surfaces of the absorbent core. The two sheets along with the back sheet are used to provide leakage-protecting baffles, which extend above the top surface of the absorbent core. In this design, there is a risk of exudates leaking out once such extended baffles twist due to body movement.

U.S. Pat. No. 5,167,653 discloses a disposable garment provided with a plurality of flaps extending along laterally opposite sides of the garment and normally biased by their own elastic shrinking potential to turn upward. This invention makes the disposable garment difficult to wear because of multiple flaps containing elastic members.

In light of the above described disadvantages, there is a need for a disposable diaper that is simple in design and provides effective prevention of leakage of body exudates. Additionally, the disposable diaper should be easy-to-wear and should prevent exudates from leaking out irrespective of body movements or posture of the person wearing the article.

### SUMMARY

The present invention improves the mechanism of blocking leakage of body exudates from a diaper by providing a unitized double cuff structure along side edges of the diaper.

In one embodiment, the disposable absorbent article provided by the invention comprises a liquid permeable top sheet, a liquid impermeable back sheet, an absorbent core interposed between the top sheet and the back sheet, and a pair of unitized double cuffs located outwardly with respect to the absorbent core along laterally opposite sides thereof.

Each cuff comprises a ribbon wherein a first portion of the ribbon is bonded to the top sheet at a predefined distance from a second portion of the ribbon thereby forming an inverted “V” structure between the attached portions. A further portion of the ribbon or fringe extension, extends upwardly beyond one of the bonds formed between the ribbon and the top sheet.

The unitized double cuff extends lengthwise all along side edges of the diaper. One or more elastic bands may be provided within the ribbon in lengthwise direction.

The above described unitized double cuff conforms tightly to a wearer's body and blocks exudates from leaking out of the diaper irrespective of the quantity of the exudates or the body posture of the wearer.

### BRIEF DESCRIPTION OF THE DRAWINGS

These and other features and advantages of the present invention will be appreciated, as they become better understood by reference to the following Detailed Description when considered in connection with the accompanying drawings:

FIG. 1 is a top plan view of an embodiment of a diaper constructed according to the present invention as being unfolded.

FIG. 2 is a cross sectional view of the diaper of the present invention.

FIG. 3 is a partial cross sectional view of the diaper of the present invention, showing a unitized double cuff.

FIG. 4 is a partial cross sectional view of a diaper of the present invention, depicting the unitized double cuff being utilized to block exudates from leaking out of the diaper.

### DETAILED DESCRIPTION OF THE INVENTION

In the foregoing specification, specific embodiments of the present invention have been described. However, one of ordinary skill in the art will appreciate that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as critical, required, or essential features or elements of any or all the claims.

With reference to the drawings, a new design for a disposable absorbent article is described with the principles and embodiments as set out below. The new design provides a unitized double cuff in the disposable absorbent article for preventing the leakage of body exudates from the disposable absorbent article such as a diaper.

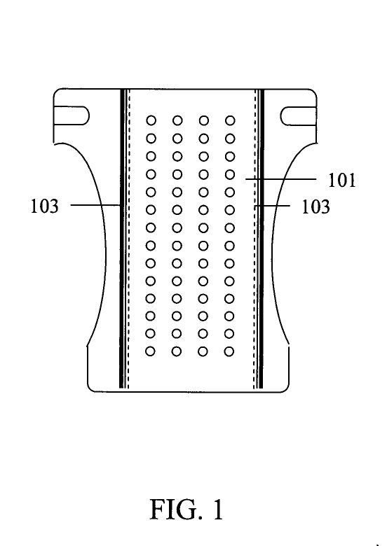

FIG. 1 shows a top plan view of an embodiment of a diaper constructed according to the present invention being unfolded. It comprises top sheet 101, leakage protecting unitized double cuff 103, and tape fasteners 104. Top sheet 101 is liquid permeable and forms the top surface of the diaper, which is in contact with a wearer's body. Body exudates pass through the topsheet onto an absorbent core [not shown in FIG. 1]. Leakage protecting unitized double cuff 103 is provided along the side edges of the top sheet and blocks the outflow of exudates from the diaper.

Top sheet 101 is preferably maintained by fibres entangled together in a configuration of a nonwoven fabric.

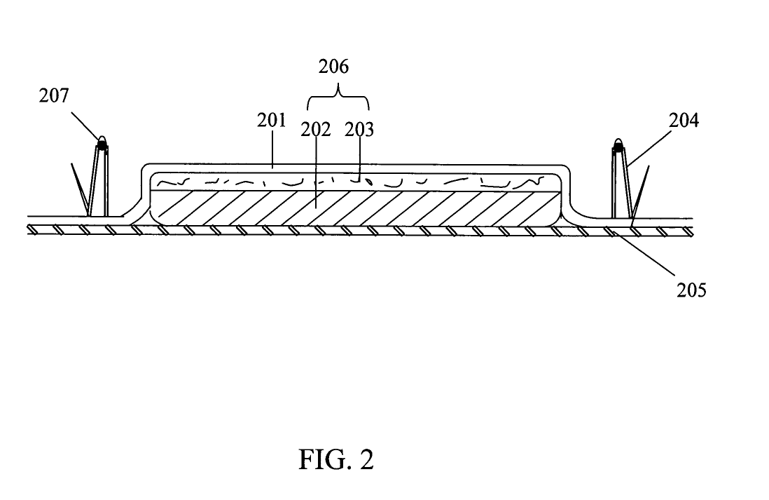

FIG. 2 shows a cross sectional view of the diaper of the present invention. It comprises top sheet 201, absorbent retaining layer 202, isolating layer 203, leakage protecting unitized double cuffs 204, elastics 207, and back sheet 205. Absorbent retaining layer 202 and isolating layer 203 together comprise absorbent core 206. Absorbent retaining layer 202 absorbs the body exudates. Isolating layer 203 is disposed between top sheet 201 and absorbent retaining layer 202, separating them from each other and allowing body exudates to pass rapidly therethrough. Isolating layer 203 not only reduces the possibility that exudates that have been absorbed by absorbent retaining layer 202 flow again through the absorbent retaining layer 202 to top sheet 201, but also gives absorbent core 206 a cushion-like nature. Leakage protecting unitized double cuffs 204 are provided near the edges of the absorbent core 206 and run lengthwise along side edges of the diaper. Back sheet 205 forms the outer surface of the diaper and is liquid-impervious.

Preferably, absorbent retaining layer 202 may be made of fluff pulp, fibrous web, or nonwoven fabric. In order to improve the absorbability of absorbent retaining layer 202, embossing and mixing of highly absorptive polymer particles is done. Isolating layer 203 is in the form of a fibrous web having a relatively low density so as to define a large vacant space therein and exhibits a high compressive elasticity recovery rate under wet conditions.

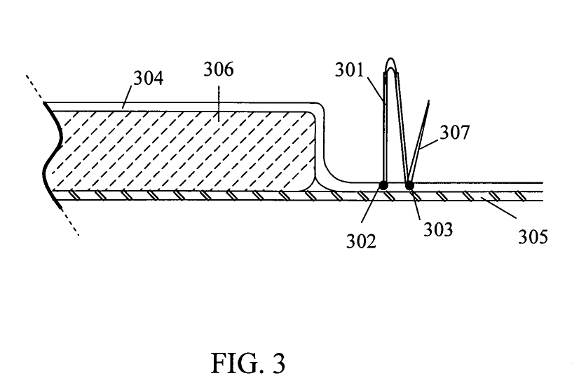

FIG. 3 is a partial cross sectional view of a diaper of the present invention showing a unitized double cuff for preventing leakage. It comprises a ribbon portion 301, a first bond 302, a second bond 303, a top sheet 304, a back sheet 305, an absorbent core 306, and a ribbon fringe extension 307. Ribbon portion 301 is bonded to top sheet 304 along its length by the first bond 302 and the second bond 303 to form an inverted “V” structure. The inverted “V” structure projects higher than the upper surface of the absorbent core 306. Fringe extension 307 of ribbon 301 can extend higher or lower than the inverted “V” and forms an angle with second bond 303.

Ribbon 301 is made of liquid impervious material such as polypropylene nonwoven. First and second bonds 302 and 303 may be manufactured using ultrasonic bonding or hot melt adhesives, which are a mixture of resins, oils, and tackifiers. The hot melt adhesive is applied, melted, and upon cooling, provides a bonding force to glue ribbon 301 to top sheet 304. Elastic bands may be provided within the inverted “V” structure lengthwise along the ribbon as well as within the fringe extension.

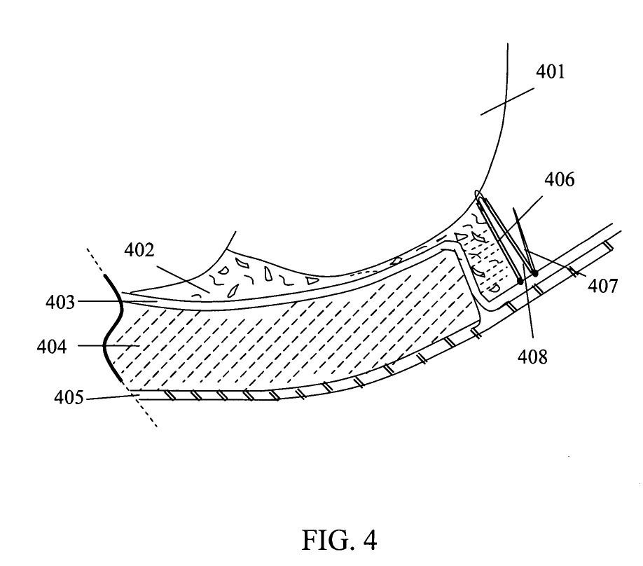

FIG. 4 is a partial cross sectional view of the diaper of the present invention, depicting the unitized double cuff being utilized to block exudates from leaking out of the diaper. It comprises a user 401, body exudates 402, a top sheet 403, an absorbent core 404, a back sheet 405, a unitized double cuff 406, including a ribbon fringe extension 407. User 401 discharges body exudates 402 onto top sheet 403. Body exudates pass through the top sheet 403 into absorbent core 404 which absorbs these exudates. However, some quantity of exudates 402 remain on top sheet 403, or flow back from absorbent core 404 to top sheet 403. These exudates flow laterally on top sheet 403 towards the sides of absorbent core 404. The exudates move from absorbent core 404 towards unitized double cuff 406. The top end of the inverted “V” of the double cuff 406 is in tight contact with body of user 401. This prevents exudates from leaking out of the diaper by flowing along the body. The fringe extension 407 of the double cuff provides a second or additional means of preventing exudates from leaking out of the diaper.

Having described the invention, it is readily apparent that various changes and modifications can be made to the above constructions without departing from the scope of the invention. Therefore, it is intended that all the matter contained in the above description should be interpreted as illustrative and not in a limiting sense. Moreover, the present invention can be used in other contexts, such as in other garments to retain exudates.

What is claimed is:

1. A disposable absorbent article comprising:
a liquid permeable top sheet;
a liquid impermeable back sheet;
an absorbent core interposed between the top sheet and the back sheet; and
a pair of unitized elasticized double cuffs located outwardly with respect to the absorbent core along laterally opposite sides thereof, each cuff comprising a ribbon bonded to the top sheet at two attachment points, wherein the first attachment point of the ribbon is located at a predefined distance from the second attachment point.

2. The disposable absorbent article as claimed in claim 1, wherein the ribbon forms an inverted “V” shaped structure.

3. The disposable absorbent article as claimed in claim 1, wherein the ribbon extends beyond the second attachment point of the ribbon and the top sheet for form a fringe extension.

4. The disposable article as claimed in claim 3, wherein one or more elastic bands are provided within the inverted “V” of the ribbon, the elastic band running lengthwise along the length of the ribbon.

5. The disposable article as claimed in claim 4, wherein one or more elastic bands are provided within the ribbon fringe extension.

6. The disposable article as claimed in claim 1, wherein the ribbon is bonded to the top sheet by hot melt adhesives or ultrasonic bonding.
